(19) United States
(12) Patent Application Publication
(10) Pub. No.: US 2009/0032021 A1
(43) Pub. Date: Feb. 5, 2009

(54) Jet-Endoscope

(76) Inventors: Dominik Lirsch, Bruckneudorf (AT); Robert Kolbl, Tulln (AT)

Correspondence Address: NIXON & VANDERHYE, PC, 901 NORTH GLEBE ROAD, 11TH FLOOR, ARLINGTON, VA, 22203, US

(21) Appl. No.: 12/086,431

(22) PCT Filed: Dec. 18, 2006
(86) PCT No.: PCT/AT2006/000522

§ 371 Date: Jun. 12, 2008

(30) Foreign Application Priority Data
Dec. 16, 2005 (AT) A 2018-2005

(51) Int. Cl. A61M 16/00; A61B 5/08; A61M 16/04

(52) U.S. Cl. 128/204.21; 600/532; 128/204.24; 128/204.23

(57) ABSTRACT

In a jet endoscope (1) for respiration and interventions in surgical applications, including connections for gas lines to which different respiratory gases with different pulsation frequencies can be connected, at least four connections are provided for at least two channels (11,12) arranged in a relatively offset manner in the axial direction of the jet endoscope (1) while opening at acute angles with respect to the axis (8) for the injection of gases with different pulsation frequencies, and at least two further channels (13,14) provided distally to the respiratory channels (11,12) for a separate gas analysis and a separate pressure measurement, respectively.

The invention relates to a jet endoscope for respiration and interventions in surgical applications, including connections for gas lines to which different respiratory gases with different pulsation frequencies can be connected.

Jet endoscopes of the initially defined kind, as a rule, comprise a tube to which connections for respiratory gases or oxygen and/or CO₂-measuring means can be connected. Known devices of the initially defined kind, besides a main axis, include connections opening into a T-bar on their distal end and ending in the interior of the tube, for instance, of a laryngoscope. In this respect, U.S. Pat. No. 5,752,506 A and U.S. Pat. No. 5,165,398 A disclose respiratory devices with superposed high-frequency respiration, which, for instance, also enable the taking of an airway pressure via a special adapter.

GB 2063686 A and DE 2947659 A1 describe high-frequency respiratory systems in which a measuring line is provided for taking respiratory gas samples. DE 2847681 A, for instance, describes a tracheal cannula into whose distal end two or three nozzles open, with a measuring line being associated to one of said nozzles.

The invention aims to provide a jet endoscope of the initially defined kind, which, in addition to enabling the intraoperative high-frequency respiration of a patient during an invasive intervention in surgical applications, also renders feasible the monitoring of the relevant respiratory gas parameters, thus substantially facilitating the continuous adaptation of the respectively required respiratory measures and permitting undistorted measurements.

To solve this object, the jet endoscope according to the invention is essentially characterized in that at least four connections are provided for at least two channels arranged in a relatively offset manner in the axial direction of the jet endoscope while opening into nozzles at acute angles with respect to said axis for the injection of gases with different pulsation frequencies, and at least two further channels provided distally to the respiratory channels for a separate gas analysis and a separate pressure measurement, respectively. The axially offset mouths of the respiratory channels for supplying respiratory gases with different pulsation frequencies first of all ensure the optimum superposition of high- and normofrequent respiratory rates while, at the same time, keeping an accordingly large clear cross-section for the introduction of medical instruments for invasive applications. By additionally providing at least two further channels distally to the respiratory channels for a separate gas analysis and a separate pressure measurement, respectively, it has become feasible to perform a gas analysis irrespectively of distortions caused by pressure measurements and, vice versa, to effect pressure measurements without distortion by the removal of gases for the gas analysis. In both cases, the airway pressure and the oxygen and/or CO₂ situation can, thus, be permanently monitored at the distal end, and the respiratory parameters as adjusted via the two nozzles located closer to the proximal end can constantly be adapted to the respective requirements. The separation of the channel for taking the airway pressure from the probe for taking gases for oxygen and/or CO₂ measurements has, thus, led to the desired improvement in the correct monitoring of the respiratory situation while permitting the continuous control of the relevant respiratory parameters.

Advantageously, the configuration according to the invention is devised such that the channel for pressure measurement opens at a distance from the channel for gas analysis measurement and axially between the respiratory channels and the channel for gas analysis. Such an arrangement of the channel for pressure measurement will result in particularly precise measuring values, which can be utilized for controlling the respiratory parameters.

Basically, the proximal end of such a jet endoscope is left open so as to enable the suction of ambient air along with the normofrequently or high-frequently injected respiratory gases. The respective channel must have an accordingly large cross-section at the proximal end of the jet endoscope in order to enable the unhindered introduction of surgical instruments. In accordance with the invention, the configuration is advantageously further developed such that at least one further connection for respiratory gas conditioning is provided on the proximal, open end of the jet endoscope, which further connection opens at an acute angle with respect to the axis of the jet endoscope. Such an additional channel opening at an acute angle with respect to the axis of the jet endoscope permits the supply of humidified air or otherwise conditioned air without affecting the clear cross-section for the introduction of surgical instruments.

A reduced adverse effect on the clear cross-section will be ensured while, at the same time, providing an accordingly high mechanical stability in that the channels provided for the connections are configured as lines extending on the outer wall of the tube in the substantially axial direction of the jet endoscope and whose distal ends each open in respectively different radial planes.

In order to be able to perform a completely unaffected and error-free measurement and gas analysis while, at the same time, effecting respiration at different pulsation frequencies, the configuration according to the invention is preferably devised such that the axial distance between the mouths of the respiratory gases and the mouths of the channels for gas analysis and pressure measurement is at least 40 mm and, preferably, at least 50 mm. In order to enable the simultaneous realization of a gas analysis and an error-free pressure measurement, the configuration is advantageously devised such that the mouths of the channels for gas analysis and separate pressure measurement, which are arranged to be offset in the axial direction by at least 5 mm, are also arranged to be offset in the peripheral direction of the tube jacket. With this configuration, it is to be taken into account that gas amounts in the order of approximately 200 ml/min will be withdrawn for the purposes of a gas analysis and, in particular, the determination of O₂ and CO₂. The airway pressure will then be determinable in a particularly error-free manner at the mouth of the connection for pressure measurement, which is offset both in the peripheral direction and in the axial direction, if said mouth is, at the same time, flushed with a reduced amount of respiratory gas. In the region closer to the distal end, of the mouth of the lines for the respiratory gases, a corresponding negative pressure would form at substantially larger injected gas amounts, which would again falsify airway pressure measurements. The minimum distances provided by the invention permit decoupling of the individual factors of influence which would distort pressure measurements.

In the following, the invention will be explained in more detail by way of an exemplary embodiment of the jet endoscope according to the invention, which is schematically illustrated in the drawing. Therein,

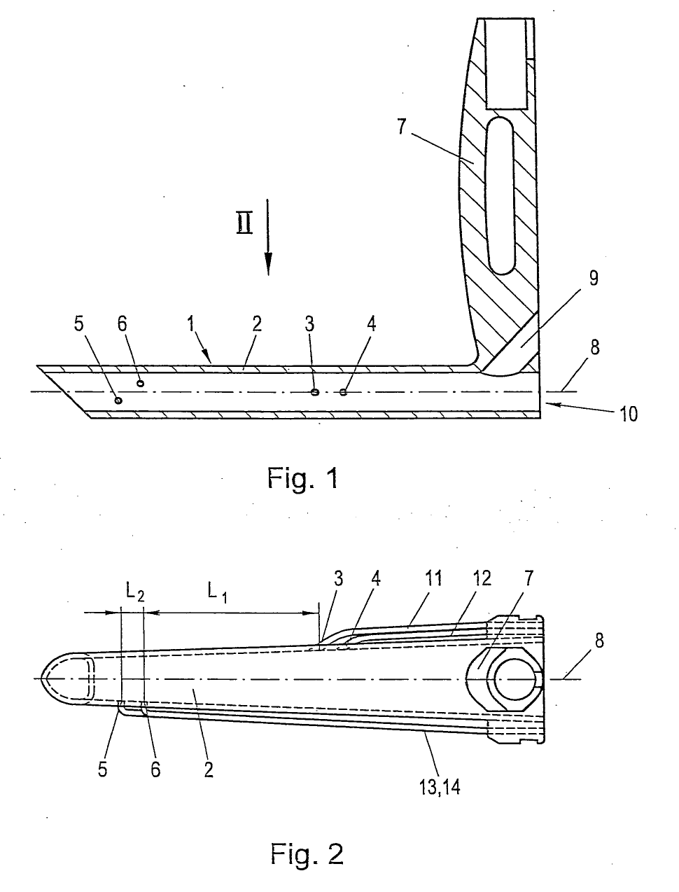

FIG. 1 is an axial section through the jet endoscope; and

FIG. 2 is a view in the sense of arrow II of FIG. 1, including details for the mouths of the individual lines.

In FIG. 1, a jet endoscope is denoted by 1, whose tube 2 comprises mouths for respiratory gases, which are schematically denoted by 3 and 4, as well as mouths for airway pressure measurement and gas analysis, which are denoted by 5 and 6, respectively. Furthermore, a grip end 7 is apparent, wherein a further connection 9 for the supply of conditioned and humidified respiratory air is provided at an acute angle with respect to the direction of the axis 8 of the tube. This humidified respiratory air may be used for respiration in addition to the air sucked in through the proximal, open end 10. The proximal, open end 10 also serves for the introduction of surgical instruments. In the illustration according to FIG. 1, low-frequency jet gas is injected via the mouth position indicated by 4, while at the position denoted by 3 the injection of high-frequency jet gas takes place, with the respective suction pressure conditions adjusting in the interior of the tube 2 in the vicinity of the mouths designed as jet nozzles.

In FIG. 2, the respective lines leading to the mouth positions denoted by 3 and 4 are denoted by 11 and 12, respectively.

FIG. 2, moreover, depicts lines 13 and 14 leading to a gas analysis measuring instrument and a pressure measuring instrument. While channels 11 and 12 may be arranged one beside the other in the plane of projection of FIG. 2, channels 13 and 14 are arranged one above the other, since the mouth positions 5 and 6 of these channels are arranged to be offset in the peripheral direction, as is schematically indicated in FIG. 1. The distance between the mouths 5 and 6 is at least 5 mm and denoted by L₂. The respective distance L₁ between the mouth 6 and the consecutive mouth 3, viewed in the axial direction towards the proximal end, is at least 40 mm. The gas amount required for the gas analysis is sucked in via the mouth 5 located closer to the distal end of the tube 2. The mouth 6, which is arranged in a manner offset in the peripheral direction and in the axial direction at said minimum distance L₂ serves to measure the airway pressure.

1. A jet endoscope for respiration and interventions in surgical applications, including connections for gas lines to which different respiratory gases with different pulsation frequencies can be connected, characterized in that at least four connections are provided for at least two channels (11,12) arranged in a relatively offset manner in the axial direction of the jet endoscope (1) while opening at acute angles with respect to said axis (8) for the injection of gases with different pulsation frequencies, and at least two further channels (13,14) provided distally to the respiratory channels (11,12) for a separate gas analysis and a separate pressure measurement, respectively.

2. A jet endoscope according to claim 1, characterized in that the channel (13) for pressure measurement opens at a distance (L₂) from the channel (14) for gas analysis measurement and axially between the respiratory channels (11,12) and the channel (14) for gas analysis.

3. A jet endoscope according to claim 1, characterized in that at least one further connection (9) for respiratory gas conditioning is provided on the proximal, open end of the jet endoscope (1), which further connection opens at an acute angle with respect to the axis (8) of the jet endoscope (1).

4. A jet endoscope according to claim 1, characterized in that the channels (11,12,13,14) provided for the connections are configured as channels extending on the outer wall of the tube in the substantially axial direction of the jet endoscope (1) and whose distal ends each open in respectively different radial planes.

5. A jet endoscope according to claim 1 4, characterized in that the axial distance between the mouths (3,4) of the respiratory gases and the mouths (5,6) of the channels (13,14) for gas analysis and pressure measurement is at least 40 mm and, preferably, at least 50 mm.

6. A jet endoscope according to claim 1, characterized in that the mouths (5, 6) of the channels (13,14) for gas analysis and separate pressure measurement, which are arranged to be offset in the axial direction by at least 5 mm, are also arranged to be offset in the peripheral direction of the tube jacket.
